(19) United States
(12) Patent Application Publication
(10) Pub. No.: US 2015/0164756 A1
(43) Pub. Date: Jun. 18, 2015

(54) Surfactant Solutions Containing N-Methyl-N-C8-C10-Acylglucamines And N-Methyl-N-C12-C14-Acylglucamines

(71) Applicants: Peter Klug, Grossostheim (DE); Franz-Xaver Scherl, Burgkirchen (DE); Carina Mildner, Frankfurt am Main (DE); Eva-Maria Keitzl, Muehldorf (DE)

(72) Inventors: Peter Klug, Grossostheim (DE); Franz-Xaver Scherl, Burgkirchen (DE); Carina Mildner, Frankfurt am Main (DE); Eva-Maria Keitzl, Muehldorf (DE)

(73) Assignee: Clariant Finance (BVI) Limited, Tortola (VG)

(21) Appl. No.: 14/403,072

(22) PCT Filed: May 29, 2013
(86) PCT No.: PCT/EP2013/061045

§ 371 Date: Nov. 21, 2014

(30) Foreign Application Priority Data
May 30, 2012 (DE) 10 2012 010 702.1

(51) Int. Cl. A61K 8/42; A61Q 5/02; A61Q 19/10; A61K 8/34

(52) U.S. Cl. CPC A61K 8/42; A61K 8/345; A61Q 5/02; A61Q 19/10; A61K 2800/596

(57) ABSTRACT

The invention relates to a surfactant solution containing (a) 28 to 80 wt.-% of a mixture of (a1) 5 to 20 wt.-% of N-methyl-N—C₈-C₁₀-acylglucamines, (a2) 50 to 95 wt.-% of N-methyl-N—C₁₂-C₁₄-acylglucamines, components (a1) and (a2) adding up to 100 wt.-%, (b) 0 to 20 wt.-% of one or more alcohols, (c) 20 to 72 wt.-% of water, and (d) 0 to 5 wt.-% of additives, components (a), (b), (c) and (d) adding up to 100 wt.-%.

The invention relates to surfactant solutions comprising N-methyl-N—C₈-C₁₀-acylglucamines and N-methyl-N—C₁₂-C₁₄-acylglucamines, and to compositions comprising these N-methyl-N-acylglucamines.

It is known that short-chain sugar surfactants can be used as solubilizers or surfactants in cleaning products and cosmetic and dermatological compositions.

WO 95/17880 discloses a hair shampoo composition comprising alkyl glycol ether sulfates and alkyl sulfates, and also polyhydroxyalkyl fatty acid amides. One alkyl glycol ether sulfate mentioned is lauryl triethylene glycol ether sulfate; one alkyl sulfate mentioned is lauryl sulfate. Compounds listed as polyhydroxyalkyl fatty acid amides are those of the formula

R²—CO—NR¹—Z

where R¹ is preferably C₁-C₄-alkyl, especially methyl, R² is preferably straight-chain C₇-C₁₉-alkyl or -alkenyl, especially straight-chain C₁₁-C₁₆-alkyl or -alkenyl, and Z is especially 1-deoxyglucityl, 2-deoxyfructityl, 1-deoxymaltityl, 1-deoxylactityl, 1-deoxygalactityl, 1-deoxymannityl or 1-deoxymaltotriothityl. The examples disclose hair shampoo compositions comprising ammonium lauryl sulfate, ammonium lauryl triethylene glycol sulfate and lauryl-N-methylglucamine.

WO 95/19415 discloses mixtures of a) N-acyl-N-methylglucamines having a C₈-C₁₀-acyl radical and b) N-acyl-N-methylglucamines having a C₁₂-C₁₈-acyl radical, especially a C₁₂-C₁₄-acyl radical. Components a) and b) are present in the mixtures in an a) to b) ratio of 80:20 to 20:80, especially in a ratio of 25:75 to 40:60. The surfactant mixtures are said to have improved surface-active properties and may contain further anionic, nonionic, cationic and amphoteric surfactants. Use in hair shampoos, hair lotions and foam baths is disclosed.

Glucamines are solids having melting points of about 85° C. (C₁₂-C₁₄-acylglucamines, containing 10% by weight of propylene glycol) and about 85° C. (C₁₆-C₁₈-acylglucamines, containing 20% by weight of propylene glycol). The corresponding pure substances actually have significantly higher melting points. C₁₂-C₁₄-Acylglucamines generally form sparingly water-soluble gels on dilution with water. Thus, the dilution of the N-acyl-N-methylglucamines in water down to 2% by weight solutions leads to the formation of a gel phase, which greatly complicates the handling of these surfactants.

N-Methyl-N-acylglucamines have the formula (I)

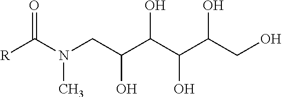

in which R is an alkyl radical or a mono- or polyunsaturated alkenyl radical.

The problem addressed by the invention is that of providing concentrated surfactant solutions comprising acylglucamines which do not cause any gel formation on dilution with water.

The problem is solved by a surfactant solution comprising

(a) 28% to 80% by weight of a mixture of
(a1) 5% to 50% by weight of N-methyl-N—C₈-C₁₀-acylglucamines,
(a2) 50% to 95% by weight of N-methyl-N—C₁₂-C₁₄-acylglucamines,
where the sum total of components (a1) and (a2) is 100% by weight,
   (b) 0% to 20% by weight of one or more alcohols,
   (c) 20% to 72% by weight of water,
   (d) 0% to 5% by weight of additives,
   where the sum total of components (a), (b), (c) and (d) is 100% by weight.

It has been found that a mixture of N-methyl-N—C₈-C₁₀-acylglucamines and N-methyl-N—C₁₂-C₁₄-acylglucamines does not cause any gel formation on dilution with water. This means that dilute solutions produced from the inventive surfactant solutions can more easily be mixed and homogenized with the further ingredients of cosmetic compositions in the formulation process. The cosmetic compositions obtained do not have any inhomogeneities. Moreover, the stirring times in the production of the cosmetic compositions are reduced.

The inventive surfactant solutions generally have a melting point of <40° C. and are therefore pumpable in the warmed state and easy to handle on the industrial scale.

The N-methyl-N-acylglucamines present in the inventive surfactant solutions contain 5% to 50% by weight of N-methyl-N-acylglucamines containing a C₈-C₁₀-acyl group. Preferably, the proportion of N-methyl-N-acylglucamines containing a C₈-C₁₀-acyl group is 11% to 19% by weight, more preferably 12% to 18% by weight. In addition, the N-methyl-N-acylglucamines contain 50% to 95% by weight of N-methyl-N-acylglucamines containing a C₁₂-C₁₄-acyl group. Preferably, the proportion of N-methyl-N-acylglucamines containing a C₁₂-C₁₄-acyl group is 81% to 89% by weight, more preferably 82% to 88% by weight.

Compositions containing 12% to 18% by weight of C₈-C₁₀-acylglucamines, based on the total content of acylglucamines, are particularly preferred since, on the one hand, gel formation is suppressed but also, on the other hand, the thickening performance of the surfactant mixtures is not reduced too greatly.

The N-methyl-N-acylglucamines may, as described in EP 0 550 637 B1, be prepared by reacting the corresponding fatty acid esters or fatty acid ester mixtures with N-methylglucamine in the presence of a solvent having hydroxyl groups or alkoxy groups. Suitable solvents are, for example, C₁-C₄ monoalcohols, ethylene glycol, 1,2-propylene glycol, glycerol and alkoxylated alcohols. Preference is given to 1,2-propylene glycol. N-Methylglucamine can, as likewise described in EP 0 550 637 A1, be obtained by reductive amination of glucose with methylamine.

Suitable fatty acid esters which are reacted with N-methylglucamine to give N-methyl-N-acylglucamines are generally the methyl esters, which are obtained by transesterification from natural fats and oils, for example the triglycerides.

N-Methyl-N—C₈-C₁₀-acylglucamines (a1) are generally derived from caprylic acid and capric acid. N-Methyl-N—C₁₂-C₁₄-acylglucamines (a2) are generally derived from lauric acid and myristic acid.

Suitable raw materials for the preparation of the fatty acid methyl esters are, for example, coconut oil or palm oil.

In addition, the inventive surfactant solutions may comprise, as component (b), one or more alcohols. Suitable alcohols are water-miscible monoalcohols or diols. Preference is given to ethanol, 1,2-propylene glycol, glycerol, 1,3-propylene glycol and isopropanol.

In a preferred embodiment of the invention, the surfactant solutions do not include any monoalcohol.

The following may be present as additives in the inventive surfactant solutions: preservatives, complexing agents and neutralizing agents, and buffers, for example citric acid or citric salts.

In a preferred embodiment, the inventive surfactant solution comprises

(a) 30% to 70% by weight of a mixture of
(a1) 11% to 19% by weight of N-methyl-N—C₈-C₁₀-acylglucamines,
(a2) 81% to 89% by weight of N-methyl-N—C₁₂-C₁₄-acylglucamines,
where the sum total of components (a1) and (a2) is 100% by weight,
   (b) 3.0% to 17.0% by weight of one or more alcohols,
   (c) 23% to 67% by weight of water,
   (d) 0% to 2% by weight of additives,
   where the sum total of components (a), (b), (c) and (d) is 100% by weight.

The invention also provides for the use of the surfactant solutions for production of cosmetic compositions.

The production of the cosmetic compositions comprises the step of diluting the surfactant solutions with water. In general, the inventive surfactant solutions are diluted with water in a ratio of 1:1 to 1:50, preferably 1:2 to 1:10. In general, the surfactant solutions are diluted to such an extent that the final concentration of the N-methyl-N-acylglucamines is in the range from 1% to 10% by weight, preferably in the range from 2% to 5% by weight.

The invention also provides concentrated compositions comprising
(A) 28% to 75% by weight of a mixture of
   (a1) 5% to 50% by weight of N-methyl-N—C₈-C₁₀-acylglucamines,
   (a2) 50% to 95% by weight of N-methyl-N—C₁₂-C₁₄-acylglucamines,
   where the sum total of components (a1) and (a2) is 100% by weight, as component (A),
(B) 0% to 10% by weight of one or more further surfactants as component (B)
(C) 0.1% to 20% by weight of one or more alcohols as component (C),
(D) 20% to 71.9% by weight of water as component (D),
(E) 0% to 5% by weight of further auxiliaries and additives as component (E),
   where the sum total of components (A), (B), (C), (D) and (E) is 100% by weight.

The compositions preferably comprise

(A) 30% to 70% by weight of component (A),
(B) 1.0% to 10% by weight of component (B),
(C) 3% to 17% by weight of component (C),
(D) 23% to 66% by weight of component (D),
(E) 0% to 5% by weight of component (E),
where the sum total of components (A), (B), (C), (D) and (E) is 100% by weight.

The further surfactants (B) may be nonionic surfactants, anionic surfactants, cationic surfactants and/or betaine surfactants.

Useful anionic surfactants include (C₁₀-C₂₂)-alkyl and -alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, alpha-sulfo fatty acid esters, alkylbenzenesulfonates, alkylphenol glycol ether sulfonates, sulfosuccinates, sulfosuccinic monoesters and diesters, fatty alcohol phosphates, fatty alcohol ether phosphates, protein-fatty acid condensation products, alkyl monoglyceride sulfates and sulfonates, alkyl glyceride ether sulfonates, fatty acid methyl taurides, fatty acid sarcosinates, sulfosuccinates, sulforicinoleates, acylglutamates and acylglycinates.

These compounds and mixtures thereof are utilized in the form of their water-soluble or water-dispersible salts, for example the sodium, potassium, magnesium, ammonium, mono-, di- and triethanolammonium and the analogous alkylammonium salts.

In one embodiment of the invention, the compositions comprise one or more anionic surfactants from the group of the alkyl sulfates and alkyl ether sulfates.

Preferred alkyl sulfates are the C₈-C₂₀-alkyl sulfates, especially the linear C₈-C₂₀-alkyl sulfates in the form of their sodium, potassium or ammonium salts. Examples of alkyl sulfates are lauryl sulfate, cocoalkyl sulfate and tallowalkyl sulfate. Particular preference is given to lauryl sulfate.

Preferred alkyl ether sulfates are the C₈-C₂₀-alkyl ether sulfates, particular preference being given to the linear C₈-C₂₀-alkyl ether sulfates, especially the alkyl glycol ether sulfates derived from the ethoxylated fatty alcohols, in the form of their sodium, potassium or ammonium salts. Examples of alkyl ether sulfates are lauryl ether sulfate, cocoalkyl ether sulfate and tallowalkyl ether sulfate. Examples of glycol ether sulfates are lauryl triethylene glycol ether sulfate, cocoalkyl triethylene glycol ether sulfate and tallowalkyl hexaethylene glycol ether sulfate. Lauryl glycol ether sulfate is especially preferred, for example lauryl triethylene glycol ether sulfate.

Betaine surfactants contain, in the same molecule, a cationic group, especially an ammonium group, and an anionic group, which may be a carboxylate group, sulfate group or sulfonate group. Suitable betaines are alkyl betaines such as cocobetaine or fatty acid alkylamidopropyl betaines, for example cocoacylamidopropyl dimethyl betaine, C₁₂-C₁₈ dimethylamino-hexanoates or C₁₀-C₁₈ acylamidopropane dimethyl betaines.

In a preferred embodiment of the invention, the compositions comprise one or more amidopropyl betaines of the formula (I)

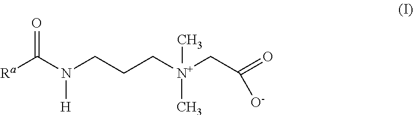

in which R^{a} is a linear or branched saturated C₇-C₂₁-alkyl group or a linear or branched mono- or polyunsaturated C₇-C₂₁-alkenyl group.

In a further preferred embodiment of the invention, the compositions comprise one or more betaines of the formula (II)

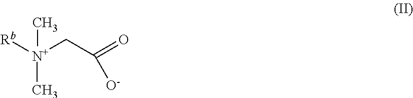

in which R^{b} is a linear or branched saturated C₈-C₂₂-alkyl group or a linear or branched mono- or polyunsaturated C₈-C₂₂-alkenyl group.

In a further preferred embodiment of the invention, the compositions comprise one or more sulfo betaines of the formula (III)

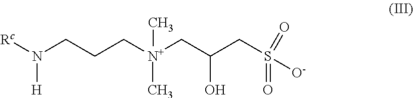

in which R^{c} is a linear or branched saturated C₈-C₂₂-alkyl group or a linear or branched mono- or polyunsaturated C₈-C₂₂-alkenyl group.

More preferably, the compositions comprise one or more betaine surfactants selected from the group of the compounds consisting of the amidopropyl betaines of the formula (I), the betaines of the formula (II) and the sulfo betaines of the formula (III).

In an especially preferred embodiment of the invention, the compositions comprise one or more betaine surfactants selected from the amidopropyl betaines of the formula (I).

In a further especially preferred embodiment of the invention, the compositions comprise one or more betaine surfactants selected from the betaines of the formula (II).

In a further especially preferred embodiment of the invention, the compositions comprise one or more betaine surfactants selected from the sulfo betaines of the formula (III).

Preferably, the R^{a} radical in the one or more amidopropyl betaines of the formula (I) is a linear or branched saturated C₇-C₁₇-alkyl group. Among the linear and branched saturated alkyl groups R^{a}, preference is given to the linear saturated alkyl groups.

More preferably, the amidopropyl betaines of the formula (I) are cocamidopropyl betaines.

Preferably, the R^{b} radical in the one or more betaines of the formula (II) is a linear or branched saturated C₈-C₁₈-alkyl group and more preferably a linear or branched saturated C₁₂-C₁₈-alkyl group. Among the linear and branched saturated alkyl groups R^{b}, preference is given to the linear saturated alkyl groups.

Preferably, the R^{c} radical in the one or more sulfo betaines of the formula (III) is a linear or branched saturated C₈-C₁₈-alkyl group and more preferably a linear or branched saturated C₁₂-C₁₈-alkyl group. Among the linear and branched saturated alkyl groups R^{c}, preference is given to the linear saturated alkyl groups.

More preferably, the compositions comprise amidopropyl betaines of the formula (I) and/or alkyl betaines of the formula (II).

Preferably, the compositions comprise, as well as the anionic surfactant, a betaine surfactant.

More preferably, the compositions comprise the above-described alkyl sulfates and/or alkyl ether sulfates and betaine surfactants.

Suitable cationic surfactants are substituted or unsubstituted, straight-chain or branched quaternary ammonium salts of the R¹N(CH₃)₃X, R¹R²N(CH₃)₂X, R¹R²R³N(CH₃)X or R¹R²R³R⁴NX type. The R¹, R², R³ and R⁴ radicals may preferably each independently be unsubstituted alkyl having a chain length between 8 and 24 carbon atoms, especially between 10 and 18 carbon atoms, hydroxyalkyl having 1 to 4 carbon atoms, phenyl, C₂- to C₁₈-alkenyl, C₇- to C₂₄-aralkyl, (C₂H₄O)ₓH where x is from 1 to 3, alkyl radicals containing one or more ester groups, or cyclic quaternary ammonium salts. X is a suitable anion. Preference is given to (C₈-C₂₂)-alkyltrimethylammonium chloride or bromide, more preferably cetyl-trimethylammonium chloride or bromide, di-(C₈-C₂₂)-alkyldimethyl-ammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, more preferably distearyldimethylammonium chloride, di-(C₈-C₂₂)-alkylamidopropyltrimethyl-ammonium chloride and methosulfate.

Examples of useful nonionic surfactants include the following compounds:
Polyethylene oxide, polypropylene oxide and polybutylene oxide condensates of alkylphenols. These compounds comprise the condensation products of alkylphenols having a C₆- to C₂₀-alkyl group which may be either linear or branched with alkene oxides.
   These surfactants are referred to as alkylphenol alkoxylates, e.g. alkylphenol ethoxylates.
Condensation products of aliphatic alcohols with 1 to 25 mol of ethylene oxide. The alkyl or alkenyl chain of the aliphatic alcohols may be linear or branched, primary or secondary, and contains generally 8 to 22 carbon atoms. Particular preference is given to the condensation products of C₁₀ to C₂₀ alcohols with 2 to 18 mol of ethylene oxide per mole of alcohol. The alcohol ethoxylates may have a narrow (“narrow range ethoxylates”) or a broad homolog distribution of the ethylene oxide (“broad range ethoxylates”).
   Examples of commercially available nonionic surfactants of this type are Tergitol® 15-S-9 (condensation product of a linear secondary C₁₁-C₁₅ alcohol with 9 mol of ethylene oxide), Tergitol® 24-L-NMW (condensation product of a linear primary C₁₂-C₁₄ alcohol with 6 mol of ethylene oxide, having narrow molar mass distribution). This product class likewise includes the Genapol® brands from Clariant.
Condensation products of ethylene oxide with a hydrophobic basis, formed by condensation of propylene oxide with propylene glycol. The hydrophobic moiety of these compounds preferably has a molecular weight between 1500 and 1800. The addition of ethylene oxide onto this hydrophobic moiety leads to an improvement in the water solubility. The product is liquid up to a polyoxyethylene content of about 50% of the total weight of the condensation product, which corresponds to a condensation with up to about 40 mol of ethylene oxide. Commercially available examples of this product class are the Pluronic® brands from BASF and the Genapol® PF brands from Clariant.
Condensation products of ethylene oxide with a reaction product of propylene oxide and ethylenediamine. The hydrophobic unit of these compounds consists of the reaction product of ethylenediamine with excess propylene oxide and generally has a molecular weight of 2500 to 3000. Ethylene oxide is added onto this hydrophobic unit up to a content of 40 to 80% by weight of polyoxyethylene and a molecular weight of 5000 to 11 000.
   Commercially available examples of this compound class are the Tetronic® brands from BASF and the Genapol® PN brands from Clariant.

Further suitable nonionic surfactants are alkyl and alkenyl oligoglycosides and fatty acid polyglycol esters or fatty amine polyglycol esters each having 8 to 20 and preferably 12 to 18 carbon atoms in the fatty alkyl radical, alkyl oligoglycosides, alkenyl oligoglycosides and fatty acid N-alkylglucamines.

In addition, the inventive compositions may comprise, as component (C), one or more alcohols which originate from the inventive surfactant solution.

Suitable alcohols are the abovementioned water-miscible alcohols. In a preferred embodiment of the invention, the compositions do not contain any monoalcohols.

Auxiliaries and additives (E) are, for example, preservatives, fragrances, dyes and refatting agents.

Suitable preservatives are preservatives listed in the relevant annex of the European cosmetics legislation, for example phenoxyethanol, benzyl alcohol, parabens, benzoic acid and sorbic acid; a particularly suitable example is 1,3-bis(hydroxymethyl)-5,5-dimethylimidazolidine-2,4-dione (Nipaguard® DMDMH).

The amount of the preservatives in the inventive compositions is generally from 0% to 2.0% by weight, based on the total weight of the finished compositions.

In a preferred embodiment of the invention, the inventive compositions are in the form of products for hair washing and skin cleansing, such as hair shampoos, shower gels, hand soaps and face cleansers.

The invention is illustrated in detail by the examples which follow.

### EXAMPLES

### Examples 1 to 4 and Comparative Example 1

The N-acyl-N-methylglucamines described hereinafter were prepared according to EP 0 550 637 from the corresponding fatty acid methyl esters and N-methylglucamine in the presence of 1,2-propylene glycol as solvent, and were obtained in solid form consisting of active substance and 1,2-propylene glycol.

| | | Active | 1,2- | Melting |
|---|---|---|---|---|
| Preparation | Methyl | substance | Propylene | point |
| example | ester | (%) | glycol (%) | (° C.) |
| 1 | C8/10 | 90 | 10 | 50 |
| 2 | C12/14 | 90 | 10 | 85 |

The above products were mixed in various ratios; the melting point of the mixture was determined. The products are difficult to handle and have melting points greater than 50° C. They were therefore diluted with water and alcohols (ethanol), so as to result in compositions which were handleable in liquid form below 50° C.

| | | | | | | Melting point | | | |
|---|---|---|---|---|---|---|---|---|---|
| | Ratio | Active | | | | of the | | | |
| | C8/10: | content of | | | propylene | surfactant | 10% in | 5% in | 2% in |
| Example | C12/14 | glucamines | Water | Ethanol | glycol | mixture (° C.) | water | water | water |
| Comparative | 0:100 | 60 | 23 | 10 | 7 | 40 | white gel | white gel | gel/liquid |
| example 1 | | | | | | | | | |
| Comparative | 10:90 | 60 | 23 | 10 | 7 | 37 | white gel | gel/liquid | gel/liquid |
| example 2 | | | | | | | | | |
| Example 1 | 15:85 | 30 | 67 | 0 | 3 | 35 | clear liquid | clear liquid | clear liquid |
| | | | | | | | about 100 | | |
| | | | | | | | mPas | | |
| Example 2 | 15:85 | 60 | 23 | 10 | 7 | 33 | clear liquid | clear liquid | clear liquid |
| | | | | | | | about 100 | | |
| | | | | | | | mPas | | |
| Example 3 | 20:80 | 60 | 23 | 10 | 7 | 30 | clear liquid | clear liquid | clear liquid |
| | | | | | | | about 100 | | |
| | | | | | | | mPas | | |
| Example 4 | 50:50 | 60 | 23 | 10 | 7 | 25 | clear liquid | clear liquid | clear liquid |

The inventive compositions, in contrast to comparative example 1, have good handleability on the one hand, and on the other hand are dilutable in water easily without gel formation. Examples 1 and 2 have the advantage over examples 3 and 4 that the concentrates have a higher thickening performance in cosmetic formulations.

1. A surfactant solution comprising
(a) 28% to 80% by weight of a mixture of
(a1) 5% to 50% by weight of N-methyl-N—C₈-C₁₀-acylglucamines,
(a2) 50% to 95% by weight of N-methyl-N—C₁₂-C₁₄-acylglucamines,
where the sum total of components (a1) and (a2) is 100% by weight,
(b) 0% to 20% by weight of one or more alcohols,
(c) 20% to 72% by weight of water,
(d) 0% to 5% by weight of additives,
where the sum total of components (a), (b), (c) and (d) is 100% by weight.

2. The surfactant solution as claimed in claim 1, wherein the weight ratio of components (a1) to (a2) is 11:19 to 81:89.

3. The surfactant solution as claimed in claim 1 or 2, comprising, as component (b), one or more alcohols selected from the group consisting of ethanol, 1,2-propylene glycol, 1,3-propylene glycol, glycerol and isopropanol.

4. The surfactant solution as claimed in any of claims 1 to 3, comprising
(a) 30% to 70% by weight of a mixture of
(a1) 11% to 19% by weight of N-methyl-N—C₈-C₁₀-acylglucamines,
(a2) 81% to 89% by weight of N-methyl-N—C₁₂-C₁₄-acylglucamines,
where the sum total of components (a1) and (a2) is 100% by weight,
(b) 3% to 17% by weight of one or more alcohols,
(c) 23% to 67% by weight of water,
(d) 0% to 2% by weight of additives,
where the sum total of components (a), (b), (c) and (d) is 100% by weight.

5. The surfactant solution as claimed in any of claims 1 to 4, which does not comprise any monoalcohol.

6. The use of a surfactant solution as claimed in any of claims 1 to 5 for production of cosmetic compositions.

7. A process for producing cosmetic compositions, comprising the step of diluting the surfactant solution as claimed in any of claims 1 to 5 with water.

8. A composition comprising
(A) 28% to 75% by weight of a mixture of
(a1) 5% to 50% by weight of N-methyl-N—C₈-C₁₀-acylglucamines,
(a2) 50% to 95% by weight of N-methyl-N—C₁₂-C₁₄-acylglucamines,
where the sum total of components (a1) and (a2) is 100% by weight, as component (A),
(B) 0% to 10% by weight of one or more further surfactants as component (B),
(C) 0.1% to 20% by weight of one or more alcohols as component (C),
(D) 20 to 71.9 of water as component (D),
(E) 0% to 5% by weight of further auxiliaries and additives as component (E),
where the sum total of components (A), (B), (C), (D) and (E) is 100% by weight.

9. The composition as claimed in claim 8, comprising
(A) 30% to 70% by weight of component (A),
(B) 1.0% to 10% by weight of component (B),
(C) 3% to 17% by weight of component (C),
(D) 23% to 66% by weight of component (D),
(E) 0% to 5% by weight of component (E).
